(19) United States
(12) Patent Application Publication
(10) Pub. No.: US 2021/0033599 A1
(43) Pub. Date: Feb. 4, 2021

(54) INFORMATION PROCESSING APPARATUS, CONTROL METHOD, AND PROGRAM

(71) Applicants: NEC CORPORATION, Tokyo (JP); NATIONAL CANCER CENTER, Tokyo (JP)

(72) Inventors: Tomoharu KIYUNA, Tokyo (JP); Yoshiko YOSHIHARA, Tokyo (JP); Hiroyasu SAIGA, Tokyo (JP); Noriko MOTOI, Tokyo (JP); Hiroshi YOSHIDA, Tokyo (JP); Yuichiro OHE, Tokyo (JP); Takashi KOHNO, Tokyo (JP)

(73) Assignees: NEC CORPORATION, Tokyo (JP); NATIONAL CANCER CENTER, Tokyo (JP)

(21) Appl. No.: 17/044,427

(22) PCT Filed: Apr. 25, 2019
(86) PCT No.: PCT/JP2019/017659

§ 371 Date: Oct. 1, 2020

(30) Foreign Application Priority Data
Apr. 26, 2018 (JP) 2018-085540

(51) Int. Cl. G01N 33/534; C12Q 1/6886

(52) U.S. Cl. CPC G01N 33/534; C12Q 2600/158; G01N 2800/52; C12Q 1/6886

(57) ABSTRACT

An information processing apparatus (2000) extracts a histomorphological feature of a tissue included in pathological image data (10). The information processing apparatus (2000) generates prediction data (30) based on the extracted histomorphological feature. The prediction data (30) indicates one or more of a prediction relating to an effect of a cancer therapeutic drug on a target patient and a prediction of a side effect of the cancer therapeutic drug on the target patient.

### TECHNICAL FIELD

The present invention relates to image analysis of a pathological image.

### BACKGROUND ART

As one of methods that diagnoses a disease of a human or an animal, pathological diagnosis using a pathological image is performed. The pathological image is an image obtained by imaging a stained section prepared from a tissue of a body of a human or an animal with a camera or a digital slide scanner.

A technique that obtains information relating to a disease, or the like by performing image analysis on data (hereinafter, referred to as pathological image data) of a pathological image has been developed. For example, Patent Document 1 discloses a technique that computes a feature value relating to a cell nucleus from pathological image data and performs a prediction of a prognosis of a disease and a prediction of a grade of malignancy of the disease based on the computed feature value and an evaluation function. In addition, for example, Patent Document 2 discloses a technique that analyzes correlation between constituents in a cell from change in feature value of the constituents of the cell with respect to a stimulus.

### RELATED DOCUMENT

### Patent Document

[Patent Document 1] International Publication No. WO2015/040990

[Patent Document 2] International Publication No. WO2018/003063

### SUMMARY OF THE INVENTION

### Technical Problem

In the present situation, the image analysis on the pathological image data is used for restricted purposes, such as the predictions of the grade of malignancy and the prognosis of the disease and correlation analysis in the cell with respect to the stimulus. The inventors have found that the image analysis on the pathological image data can be used for other than the purposes. One object of the invention is to provide a new using method of image analysis on pathological image data.

### Solution to Problem

An information processing apparatus of the invention includes 1) an extraction unit that extracts a histomorphological feature of a tissue included in pathological image data of a target patient, and 2) a generation unit that generates prediction data indicating a prediction relating to an influence of a cancer therapeutic drug on the target patient using the extracted histomorphological feature.

A control method of the invention is a control method that is executed by a computer. The control method includes 1) an extraction step of extracting a histomorphological feature of a tissue included in pathological image data of a target patient, and 2) a generation step of generating prediction data indicating a prediction relating to an influence of a cancer therapeutic drug on the target patient using the extracted histomorphological feature.

A program of the invention causes a computer to execute each step of the control method of the invention.

### Advantageous Effects of Invention

According to the invention, a new using method of image analysis on pathological image data is provided.

### BRIEF DESCRIPTION OF THE DRAWINGS

The above-described object and other objects, features, and advantages will become apparent from preferable example embodiments described below and the accompanying drawings.

FIG. 1 is a diagram conceptually illustrating the operation of an information processing apparatus of Example Embodiment 1.

FIG. 2 is a block diagram illustrating the functional configuration of the information processing apparatus.

FIG. 3 is a diagram illustrating a computer for implementing the information processing apparatus.

FIG. 4 is a flowchart illustrating a flow of processing that is executed by the information processing apparatus of Example Embodiment 1.

FIG. 5 is a diagram illustrating a flow of processing of extracting a histomorphological feature from pathological image data.

FIG. 6 is a diagram illustrating prediction data in a table format.

### DESCRIPTION OF EMBODIMENTS

Hereinafter, an example embodiment of the invention will be described referring to the drawings. In all drawings, the same components are represented by the same reference numerals, and description thereof will not be repeated as appropriate. In respective block diagrams, unless particular description is provided, each block is not a configuration of a hardware unit but a configuration of a function unit.

### Example Embodiment 1

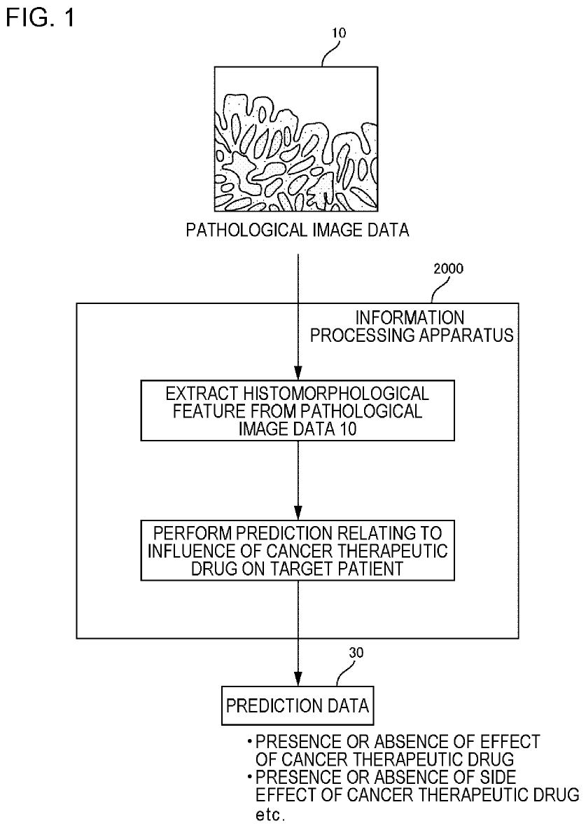

FIG. 1 is a diagram conceptually illustrating the operation of an information processing apparatus 2000 of Example Embodiment 1. Note that FIG. 1 merely shows an example of the operation for ease of understanding of the information processing apparatus 2000, and is not intended to limit the functions of the information processing apparatus 2000.

The information processing apparatus 2000 performs image analysis on pathological image data 10. The pathological image data 10 is image data obtained by imaging a tissue in a body of a human or an animal to be diagnosed (hereinafter, referred to as a target patient) with a camera. More specifically, for example, a tissue is sampled from inside of the body of the target patient, a tissue section cut from the sampled tissue is enlarged by a microscope, and the enlarged tissue is imaged by a camera, whereby the pathological image data 10 can be generated.

The information processing apparatus 2000 performs a prediction relating to an influence of a cancer therapeutic drug on the target patient based on a histomorphological feature of the tissue included in the pathological image data 10. Specifically, the information processing apparatus 2000 extracts the histomorphological feature of the tissue included in the pathological image data 10 and generates prediction data 30 based on the extracted histomorphological feature. The prediction data 30 is information indicating the prediction relating to the influence of the cancer therapeutic drug on the target patient. Specifically, the prediction data 30 includes any one of a prediction relating to an effect of the cancer therapeutic drug on the target patient and a prediction relating to a side effect of the cancer therapeutic drug on the target patient.

### Operations and Effects

With the information processing apparatus 2000 of the example embodiment, the prediction relating to the influence of the cancer therapeutic drug on the target patient is obtained using the histomorphological feature obtained by performing the image analysis on the pathological image data 10 of the target patient. Accordingly, with the information processing apparatus 2000, the pathological image data can be used for a new prediction other than a prediction of a grade of malignancy or a prognosis of a disease.

With the use of the information processing apparatus 2000, a physician can refer to a prediction made by a computer relating to an effect or a side effect of the cancer therapeutic drug, and then, can determine whether or not to administer the cancer therapeutic drug to the target patient. Accordingly, the physician can accurately determine whether or not to administer the cancer therapeutic drug. As described below, in a case where the information processing apparatus 2000 performs a prediction relating to an effect or a side effect of each of a plurality of cancer therapeutic drugs, the physician can more accurately determine what kind of cancer therapeutic drug is appropriate for the target patient.

More accurate determination about administration of a cancer therapeutic drug has an effect of increasing a probability that cancer is cured or an effect of reducing a probability that a side effect of a cancer therapeutic drug occurs. There is also an effect that a patient can be given more accurate explanation of an effect or a side effect of the cancer therapeutic drug before administering a cancer therapeutic drug.

Hereinafter, the example embodiment will be described in more detail.

### <Example of Functional Configuration>

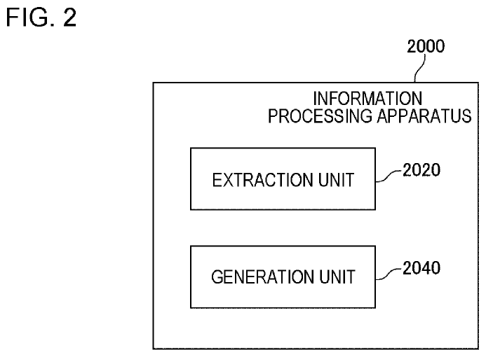

FIG. 2 is a block diagram illustrating the functional configuration of the information processing apparatus 2000. The information processing apparatus 2000 has an extraction unit 2020 and a generation unit 2040. The extraction unit 2020 extracts the histomorphological feature of the tissue included in the pathological image data 10 of the target patient. The generation unit 2040 generates the prediction data 30 using the extracted histomorphological feature.

### <Example of Hardware Configuration of Information Processing Apparatus 2000>

Each functional component of the information processing apparatus 2000 may be implemented by hardware (for example, a hard-wired electronic circuit or the like) that implements each functional component or may be implemented by a combination of hardware and software (for example, a combination of an electronic circuit and a program that controls the electronic circuit, or the like). Hereinafter, a case where each functional component of the information processing apparatus 2000 is implemented by a combination of hardware and software will be further described.

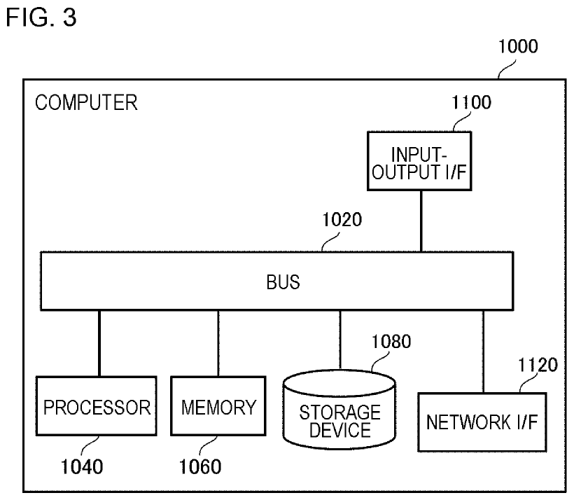

FIG. 3 is a diagram illustrating a computer 1000 for implementing the information processing apparatus 2000. The computer 1000 is any kind of computer. For example, the computer 1000 is a personal computer (PC), a server machine, a tablet terminal, a smartphone, or the like. The computer 1000 may be a dedicated computer designed in order to implement the information processing apparatus 2000 or may be a general-purpose computer.

A processor 1040 is various processors, such as a central processing unit (CPU), a graphics processing unit (GPU), and a field-programmable gate array (FPGA). A memory 1060 is a main storage that is implemented using a random access memory (RAM) or the like. A storage device 1080 is an auxiliary storage that is implemented using a hard disk, a solid state drive (SSD), a memory card, or a read only memory (ROM).

The input-output interface 1100 is an interface that connects the computer 1000 and an input-output device. For example, an input apparatus, such as a keyboard, or an output apparatus, such as a display apparatus, is connected to the input-output interface 1100. The network interface 1120 is an interface that connects the computer 1000 to a communication network. The communication network is, for example, a local area network (LAN) or a wide area network (WAN). A method in which the network interface 1120 is connected to the communication network may be wireless connection or may be wired connection.

The storage device 1080 stores a program module that implements each functional component of the information processing apparatus 2000. The processor 1040 reads each program module to the memory 1060 and executes each program module, thereby implementing a function corresponding to each program module.

### <Flow of Processing>

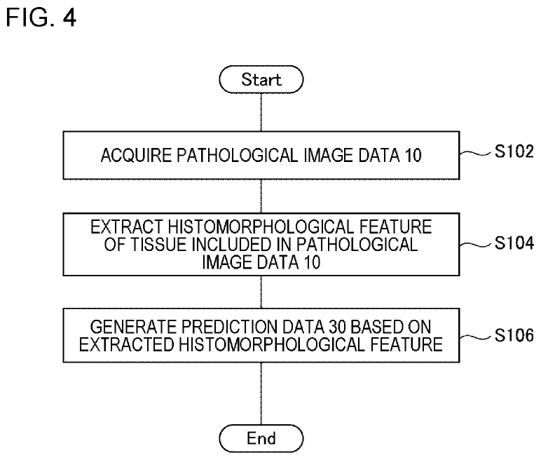

FIG. 4 is a flowchart illustrating a flow of processing that is executed by the information processing apparatus 2000 of Example Embodiment 1. The extraction unit 2020 acquires the pathological image data 10 (S102). The extraction unit 2020 extracts the histomorphological feature of the tissue included in the pathological image data 10 (S104). The generation unit 2040 generates the prediction data 30 using the extracted histomorphological feature (S106).

A timing at which the information processing apparatus 2000 executes a series of processing shown in FIG. 4 varies. For example, the information processing apparatus 2000 executes a series of processing in response to a user's operation to instruct the execution of the processing. For example, the user performs an operation to select one from among the pathological image data 10 stored in the storage apparatus. As a result, the information processing apparatus 2000 generates the prediction data 30 for the selected pathological image data 10 as a target. In addition, for example, the information processing apparatus 2000 may execute a series of processing shown in FIG. 4 in response to reception of the pathological image data 10 from an external apparatus. For example, the pathological image data 10 is transmitted from the camera that generates the pathological image data 10.

### <Cancer Therapeutic Drug>

A cancer therapeutic drug to be predicted is any drug that is used to cure cancer. For example, the cancer therapeutic drug is an immune checkpoint blockade. In addition, for example, the cancer therapeutic drug may be an anticancer agent or the like.

### <Kind of Cancer>

The information processing apparatus 2000 may predict an effect of the cancer therapeutic drug without specifying the kind of cancer or may predict an effect of a cancer therapeutic drug on a specific kind of cancer. For example, the information processing apparatus 2000 predicts an effect of a cancer therapeutic drug for a lung cancer or melanoma as a target. The kind of cancer for which the information processing apparatus 2000 can predict an effect is not limited to the above-described kinds.

### <Acquisition of Pathological Image Data 10: S102>

The information processing apparatus 2000 acquires the pathological image data 10 (S102). The pathological image data 10 may be image data generated by the camera as it is or may be image data obtained by processing image data generated by the camera. In the latter case, for example, the pathological image data 10 is generated by performing image processing (trimming) of deleting an unnecessary image region, tone correction for ease of extraction of the histomorphological feature, and the like on the image data generated by the camera. The image processing may be executed by the information processing apparatus 2000 or may be executed by an apparatus other than the information processing apparatus 2000.

In generating the pathological image data 10, a tissue section is stained by a predetermined method such that image analysis of a substance as an extraction target of the histomorphological feature is facilitated. The substance to be an extraction target of the histomorphological feature is, for example, PD-L1, an immune cell, a tumor cell, or the like as described above. In a case where a histomorphological feature is extracted for PD-L1 or an immune cell, for example, immunohistochemistry (IHC) is performed on a tissue section. In a case where a histomorphological feature is extracted for a tumor cell, for example, hematoxylin and eosin (HE) staining is performed on a tissue section. Note that IHC staining for extracting the histomorphological feature relating to PD-L1 and IHC staining for extracting the histomorphological feature relating to the immune cell are performed using different antibodies.

Here, it is assumed that histomorphological features are extracted from a plurality of kinds of substances. In this case, a plurality of tissue sections sampled from a target patient are stained by different methods, thereby generating pathological image data 10 for each substance from which the histomorphological feature is extracted. In this case, it is preferable that a plurality of tissue sections are prepared by cutting a plurality of sections from a group of tissues. In this way, it is possible to obtain a plurality of pieces of pathological image data 10 representing the substantially same tissue structure.

A method in which the information processing apparatus 2000 acquires the pathological image data 10 is any method. For example, the information processing apparatus 2000 accesses the storage apparatus, in which the pathological image data 10 is stored, thereby acquiring the pathological image data 10. The storage apparatus, in which the pathological image data 10 is stored, may be provided in the camera that generates the pathological image data 10 or may be provided outside the camera. In addition, for example, the information processing apparatus 2000 may receive the pathological image data 10 transmitted from the camera, thereby acquiring the pathological image data 10.

### <Extraction of Histomorphological Feature: S104>

The extraction unit 2020 extracts the histomorphological feature of the tissue included in the pathological image data 10 (S104). The histomorphological feature to be extracted is an image feature relating to a shape, a distribution, or the like of cells constituting the tissue or a substance, such as protein. Hereinafter, a substance from which a histomorphological feature is extracted and a histomorphological feature that extracted from the substance will be described in connection with a specific example.

### <<Histomorphological Feature Relating to PD-L1>>

PD-L1 is a molecule that is expressed in a tumor cell or the like, and is bonded to a PD-1 molecule of an immune cell, thereby suppressing the activity of the immune cell. Accordingly, in a case where PD-L1 is much expressed, the activity of the immune cell is largely suppressed. From this point, it can be said that PD-L1 is a substance closely related to recovery of cancer. For this reason, it is considered that the histomorphological feature relating to PD-L1 and the influence of the cancer therapeutic drug on the target patient are correlated. In particular, it is considered that the effect of the immune checkpoint blockade is highly correlated with the histomorphological feature relating to PD-L1. This is because the immune checkpoint blockade is a medicine that is, instead of PD-1 of the immune cell, bonded to PD-L1 of the tumor cell such that the activity of the immune cell is not suppressed.

Now, for example, the extraction unit 2020 extracts the histomorphological feature of PD-L1 included in the pathological image data 10. For example, the extraction unit 2020 extracts, as the histomorphological feature, one or more of a positive rate, an index value (entire circumference of PD-L1 with respect to a tumor cell) representing to what degree PD-L1 surrounds the tumor cell, a degree (staining intensity of PD-L1) to which PD-L1 is stained, and the size of a tumor cell with expression of PD-L1.

The positive rate is a rate of a cell positive for expression of a molecule to be stained with respect to all evaluation targets. For example, in regard to PD-L1, it is defined that “Stainability in a cell membrane of a target tumor cell is set as a target for evaluation, and a tumor proportion score (TPS, a rate of PD-L1-positive cells with respect to all tumor cells) is used as an index. Regardless of staining intensity or whether staining of the cell membrane is partial or entirely circumferential, in a case where a tumor cell is stained even a little, determination is made that the tumor cell is positive”. Now, for example, the positive rate of PD-L1 is computed as a rate with the total number of tumor cells as a denominator and the number of tumor cells with expression of PD-L1 as a numerator.

The entire circumference of PD-L1 with respect to the tumor cell is represented by, for example, a ratio of the total length of stained portions in the cell membrane of the tumor cell to the length of the entire cell membrane. The staining intensity of PD-L1 is represented by, for example, a ratio of a statistic (for example, an average value) of brightness of pixels representing PD-L1 in the pathological image data 10 to reference brightness. The pixels representing PD-L1 are pixels representing the stained portions in the pathological image data 10. The reference brightness is the brightness of PD-L1 in a case where staining is the strongest. The size of the tumor cell with expression of PD-L1 is represented by, for example, a distance between the center of a cell nucleus of the tumor cell and the cell membrane of the tumor cell.

In order to compute the entire circumference of PD-L1 with respect to the tumor cell or the size of the tumor cell with expression of PD-L1, there is a need to detect the tumor cell from the pathological image data 10. A method of detecting a tumor cell from the pathological image data 10 will be described below.

The entire circumference of PD-L1 with respect to the tumor cell or the size of the tumor cell with expression of PD-L1 is computed for a plurality of tumor cells. For example, the extraction unit 2020 extracts a statistic of index values computed for a plurality of tumor cells as a histomorphological feature relating to PD-L1. For example, the extraction unit 2020 computes the entire circumference of PD-L1 for a plurality of tumor cells and sets a statistic (for example, an average value) of a plurality of computed values as the entire circumference of PD-L1 extracted from the pathological image data 10. The same applies to the size of the tumor cell. Note that the index value, such as the entire circumference, may be computed for all detected tumor cells or may be compute for a part of detected tumor cells.

### <<Histomorphological Feature Relating to Immune Cell>>

Since an immune cell (in particular, a CD4-positive T cell or a CD8-positive T cell) has a function of excluding a tumor cell, it can be said that the immune cell is a substance closely related to recovery of cancer. For this reason, it is considered that a histomorphological feature relating to the immune cell is correlated with the influence of the cancer therapeutic drug on the target patient.

Now, for example, the extraction unit 2020 extracts a histomorphological feature for the immune cell (for example, one or both of a CD4-positive T cell and a CD8-positive T cell) included in the pathological image data 10. For example, the extraction unit 2020 extracts one or more of a positive rate, a degree (staining intensity of an immune cell) to which an immune cell is stained, the size of an immune cell, and a distribution of immune cells as a histomorphological feature.

The positive rate of the immune cell can be computed as a rate based on the number of cells, for example, similarly to the positive rate of PD-L1. In addition, for example, the positive rate of the immune cell may be computed with a tumor tissue area as a denominator and an area of the immune cell as a numerator.

Ways of representing the staining intensity and the size of the immune cell are the same as the ways of representing the staining intensity and the size of PD-L1.

The size of the immune cell is computed for a plurality of immune cells. In this respect, the extraction unit 2020 extracts a histomorphological feature representing the size of the immune cell similarly to the histomorphological feature representing the size of the tumor cell computed for a plurality of tumor cells with expression of PD-L1.

The distribution of the immune cells is an index representing a distribution of positions of the immune cells in the pathological image data 10. For example, the distribution of the immune cells represents a degree to which the immune cells disperse in the entire pathological image data 10. For example, in this case, the extraction unit 2020 divides an image region of the pathological image data 10 into a plurality of partial regions and counts the number of immune cells included in each partial region. In this way, a histogram representing the number of immune cells included in each partial region can be obtained, and the distribution of the immune cells is represented by the histogram.

In addition, for example, the distribution of the immune cells may be a distribution defined by a positional relationship between an immune cell and a tumor cell. For example, in this case, the distribution of the immune cells is computed as a rate with the total number of immune cells as a denominator and the number of immune cells positioned in a tumor cell as a numerator. In this case, in a case where information relating to a tumor cell is used for computation of the distribution of the immune cells, the extraction unit 2020 detects a tumor cell from the pathological image data 10.

### <<Histomorphological Feature Relating to Tumor Cell>>

The cancer therapeutic drug is a medicine that excludes a tumor cell directly or indirectly. For this reason, it is considered that various kinds of information relating to a tumor cell are largely related to the effect or the side effect of the cancer therapeutic drug. Accordingly, it can be said that the histomorphological feature of the tumor cell is largely correlated with the influence of the cancer therapeutic drug on the target patient.

Now, for example, the extraction unit 2020 extracts a histomorphological feature for a cell nucleus of one or more tumor cells included in the pathological image data 10. For example, for the cell nucleus of the tumor cell, the extraction unit 2020 extracts, as a histomorphological feature, one or more of an area, a perimeter, a degree of circularity (a degree close to a perfect circle), a degree of complexity of a contour, an index value relating to texture, a major diameter, a minor diameter, density, and a ratio of the area of the cell nucleus to an area of a bounding rectangle of the cell nucleus. The index value relating to the texture of the cell nucleus is, for example, an angular secondary moment, contrast, uniformity, or entropy. Note that, as a technique for extracting the histomorphological feature relating to the cell nucleus described above from image data, an existing technique can be used.

### <<Detection of Tumor Cell>>

As described above, the extraction unit 2020 detects a tumor cell from the pathological image data 10 in order to extract a histomorphological feature. As a technique for detecting a tumor cell from the pathological image data 10, an existing technique can be used. For example, a detector that is implemented by a neural network, or the like is made to learn so as to detect a tumor cell from image data. In this way, a detector that detects a tumor cell from the pathological image data 10 can be constituted. The extraction unit 2020 inputs the pathological image data 10 to the detector, thereby detecting the tumor cell from the pathological image data 10.

Here, a tumor cell is more easily detected from a HE-stained tissue section than an IHC-stained tissue section. Now, it is suitable that the extraction unit 2020 performs the detection of a tumor cell using image data of an HE-stained tissue section. For example, as described above, it is assumed that a plurality of tissue sections are cut from a group of tissues sampled from the target patient, thereby generating the pathological image data 10 of the tissue sections stained by different methods. In this case, a tissue having the substantially same structure is included in all of a plurality of pieces of pathological image data 10.

Now, first, the extraction unit 2020 performs image analysis on the pathological image data 10 of the HE-stained tissue sections, thereby detecting the tumor cell. The extraction unit 2020 regards that tumor cell detected from the pathological image data 10 of the HE-stained tissue section is also present in the pathological image data 10 stained by other methods in the same size and position, and extracts a histomorphological feature from each piece of pathological image data 10.

### <<Extraction of Region of Interest (ROI)>>

The extraction unit 2020 may extract a histomorphological feature from the entire pathological image data 10 or may extract a histomorphological feature from a partial image region of the pathological image data 10. Hereinafter, a partial image region is referred to as an “ROI”. The number of ROIs extracted from one piece of pathological image data 10 may be one or may be plural.

There are various methods of extracting an ROI from the pathological image data 10. For example, the extraction unit 2020 receives a user's operation to specify an ROI. In addition, for example, the extraction unit 2020 automatically extracts an ROI from the pathological image data 10. Hereinafter, a method of automatically extracting an ROI will be described.

First, the extraction unit 2020 executes image processing on the pathological image data 10, thereby generating image data with the center of the cell nucleus highlighted. Highlighting of the center of the cell nucleus can be implemented, for example, by applying a ring filter having the same radius as the cell nucleus on image data obtained through gray scale conversion of the pathological image data 10. Note that image data obtained by inverting a green (G) channel constituting the pathological image data 10, instead of image data obtained through gray scale conversion of the pathological image data 10, may be used.

In addition, the extraction unit 2020 searches for a peak of a brightness value for image data with the center of the cell nucleus highlighted, thereby determining a center position of each cell nucleus. Then, the extraction unit 2020 extracts, as an ROI, an image region that has predetermined shape and size and of which a center position is the center position of the cell nucleus. In this way, one ROI is extracted per cell nucleus.

However, according to this method, it is considered that an overlap between the ROIs becomes large in many cases. Now, it is suitable that the extraction unit 2020 adjusts the overlap between the ROIs to reduce the ROIs (that is, performs thinning of the ROIs). Thinning of the ROIs can be implemented, for example, as follows.

First, the extraction unit 2020 counts, for each ROI, cell nuclei within an image region of a predetermined radius d from the center of the ROI, and orders the ROIs with the number of counted cell nuclei. The extraction unit 2020 determines an ROI having the largest count number of the cell nuclei as an ROI to be not deleted. The extraction unit 2020 deletes an ROI having the center in an image region of a predetermined radius R from the center of the determined ROI. With this, since the ROI near the ROI to be not deleted is deleted, the overlap between the ROIs decreases.

In addition, the extraction unit 2020 determines an ROI having the largest count number of the cell nuclei among the remaining ROIs (excluding the ROI determined as the ROI to be not deleted) as an ROI to be not deleted, and deletes an ROI having the center in an image region of a predetermined radius R from the center of the ROI. Hereinafter, the extraction unit 2020 repeats the same processing until all of the remaining ROI become an ROI to be not deleted.

As described above, it is assumed that a plurality of tissue sections are cut from a group of tissues sampled from the target patient, thereby generating the pathological image data 10 of the tissue sections stained by different methods. In this case, the extraction unit 2020 may extract an ROI in the same position and size for a plurality of pieces of pathological image data 10. Specifically, first, the extraction unit 2020 extracts an ROI based on a user's operation or a predetermined criterion for one of a plurality of pieces of pathological image data 10.

Thereafter, the extraction unit 2020 also extracts the same ROI from other pieces of pathological image data 10. In this way, it is possible to reduce a time needed for extracting an ROI or computer resources.

### <<Example of Flow of Processing of Extracting Histomorphological Feature>>

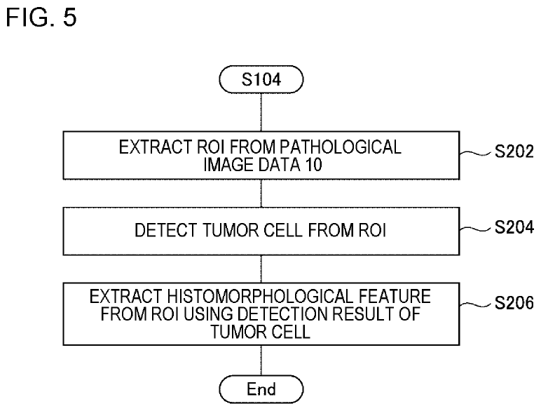

Considering the detection of the tumor cell or the extraction of the ROI described above, a flow of processing of extracting a histomorphological feature from the pathological image data 10 is, for example, as shown in FIG. 5. FIG. 5 is a diagram illustrating a flow of processing of extracting a histomorphological feature from the pathological image data 10.

The extraction unit 2020 extracts an ROI from the pathological image data 10 (S202). The extraction unit 2020 detects a tumor cell from the ROI (S204). The extraction unit 2020 extracts a histomorphological feature from the ROI using a detection result of the tumor cell (S206).

### <Generation of Prediction Data 30: S106>

The generation unit 2040 generates the prediction data 30 using the histomorphological feature extracted by the extraction unit 2020 (S106). The number of histomorphological features for use in generating the prediction data 30 may be one or may be plural. In a case where a plurality of histomorphological features are used, the histomorphological features extracted from one piece of pathological image data 10 may be used or the histomorphological features extracted from a plurality of pieces of pathological image data 10 may be used. For example, in the former case, a plurality of histomorphological features relating to PL-D1 are used. On the other hand, in the latter case, for example, one or more histomorphological features relating to PL-D1 and one or more histomorphological features relating to the immune cell are used.

The prediction data 30 indicates one or more of a prediction relating the effect of the cancer therapeutic drug on the target patient and a prediction relating to the side effect of the cancer therapeutic drug on the target patient. Hereinafter, each prediction will be described.

### <<Prediction Relating to Effect of Cancer Therapeutic Drug>>

The prediction data 30 relating to the effect of the cancer therapeutic drug indicates whether or not the cancer therapeutic drug exhibits an effect on the target patient (either one of “presence of an effect” or “absence of an effect”), a probability that the cancer therapeutic drug exhibits an effect on the target patient, the magnitude of the effect that the cancer therapeutic drug exhibits on the target patient, or the like.

There are various methods that perform the predictions using the histomorphological feature. For example, a prediction model that has learned so as to output the prediction of the effect of the cancer therapeutic drug on the target patient is used. As the prediction model, various models, such as a neural network, a support vector machine (SVM), and a decision tree, can be employed. The generation unit 2040 inputs the histomorphological feature extracted by the extraction unit 2020 to the prediction model. As a result, data representing the prediction relating to the effect of the cancer therapeutic drug is output from the prediction model.

In a case of generating the prediction data 30 indicating whether or not the cancer therapeutic drug exhibits an effect on the target patient, the prediction model outputs data representing whether or not the cancer therapeutic drug exhibits an effect on the target patient in response to the input of the histomorphological feature. For example, in a case where an effect is predicted to be exhibited, 1 is output from the prediction model, and in a case where an effect is predicted to be not exhibited, 0 is output from the prediction model. In a case of generating the prediction data 30 indicating the probability that the cancer therapeutic drug exhibits an effect on the target patient, the prediction model outputs the probability that the cancer therapeutic drug exhibits an effect on the target patient in response to the input of the histomorphological feature. In a case of generating the prediction data 30 indicating the magnitude of the effect that the cancer therapeutic drug exhibits on the target patient, the prediction model outputs data representing the magnitude of the effect that the cancer therapeutic drug exhibits on the target patient in response to the input of the histomorphological feature. The magnitude of the effect of the cancer therapeutic drug is represented by, for example, one of a predetermined number of grades of evaluation (for example, five-grade evaluation).

Learning of the prediction model is performed using training data. For example, training data is generated by a physician actually administering the cancer therapeutic drug to a patient and diagnosing a subsequent state of the patient. Specifically, training data is a combination of (1) a histomorphological feature of image data of a tissue section of the patient, and (2) data (hereinafter, referred to as result data) representing a state of the patient after the cancer therapeutic drug is administered to the patient.

In a case of predicting whether or not the cancer therapeutic drug exhibits an effect on the target patient or predicting the probability that the cancer therapeutic drug exhibits an effect on the target patient, the result data is data (for example, in a case where an effect is exhibited, 1, and in a case where an effect does not appear, 0) representing whether or not the cancer therapeutic drug exhibits an effect after the cancer therapeutic drug is administered to the patient. In a case of predicting the magnitude of the effect that the cancer therapeutic drug exhibits on the target patient, the result data is data representing to what degree the effect is large after the cancer therapeutic drug is administered to the patient. The magnitude of the effect of the cancer therapeutic drug is determined, for example, by the physician who diagnoses the patient selecting one of the predetermined number of grades of evaluation.

In a case where there are a plurality of kinds of cancer therapeutic drugs to predict an effect, a prediction model is prepared for each kind of cancer therapeutic drug. For example, training data generated for a patient to which a cancer therapeutic drug A is administered is used for learning of a prediction model that performs a prediction relating to the cancer therapeutic drug A, and training data generated for a patient to which a cancer therapeutic drug B is administered is used for learning of a prediction model that performs a prediction relating to the cancer therapeutic drug B. The generation unit 2040 inputs the histomorphological feature extracted by the extraction unit 2020 to each prediction model, thereby generating the prediction data 30 for the target patient for each cancer therapeutic drug.

### <<Prediction Relating to Side Effect>>

The prediction data 30 indicating the prediction relating to the side effect of the cancer therapeutic drug indicates, for example, whether or not the side effect occurs due to administration of the cancer therapeutic drug to the target patient (either one of “occurs” or “do not occur”), a probability that the side effect occurs due to administration of the cancer therapeutic drug to the target patient, or the magnitude of the side effect that occurs due to administration of the cancer therapeutic drug to the target patient. The prediction data 30 indicating the prediction relating to the side effect of the cancer therapeutic drug can be generated by the same method as the prediction data 30 indicating the prediction relating to the effect of the cancer therapeutic drug. For example, a prediction model that has learned so as to output the prediction relating to the side effect of the cancer therapeutic drug on the target patient is used. The generation unit 2040 inputs the histomorphological feature extracted by the extraction unit 2020 to the prediction model to obtain the prediction relating to the side effect of the cancer therapeutic drug and generates the prediction data 30 indicating the prediction.

The training data that is used for learning the prediction model, which outputs the prediction relating to the side effect of the cancer therapeutic drug, can be generated by the physician actually administering the cancer therapeutic drug to the patient and diagnosing a subsequent state of the patient, for example, similarly to the training data that is used for learning of the prediction model, which outputs the prediction relating to the effect of the cancer therapeutic drug.

In a case where there are a plurality of kinds of cancer therapeutic drugs to predict a side effect, a prediction model is prepared for each kind of cancer therapeutic drug. The generation unit 2040 inputs the histomorphological feature extracted by the extraction unit 2020 to each prediction model, thereby generating the prediction data 30 indicating the side effect of each cancer therapeutic drug on the target patient.

There may be a plurality of kinds of side effects. In this case, the generation unit 2040 may generate the prediction data 30 while distinguishing the kinds of the side effects. That is, the generation unit 2040 may generate the prediction data 30 for each kind of side effect. In this case, a prediction model is prepared for each kind of side effect. The generation unit 2040 inputs the histomorphological feature extracted by the extraction unit 2020 to each prediction model, thereby generating the prediction data 30 indicating the prediction of each side effect on the target patient.

In addition, in a case where there are a plurality of kinds of cancer therapeutic drugs to predict a side effect, a prediction model is prepared for each combination of “the kind of cancer therapeutic drug and the kind of side effect”.

### <<Histomorphological Feature for Use in Prediction>>

One or more histomorphological feature for use in a prediction may be automatically decided by the information processing apparatus 2000 or may be manually decided by a user (for example, a physician) of the information processing apparatus 2000. For example, in a case where a deep neural network is used as a prediction model (that is, in a case where the prediction data 30 is generated using deep learning), as a result of learning of the prediction model using training data, a histomorphological feature useful for a prediction is automatically decided among a plurality of histomorphological features.

On the other hand, in a case of manually deciding a histomorphological feature to be used, for example, the user, such as a physician, decides one or more histomorphological features useful for a prediction and generates a prediction model so as to generate the prediction data 30 using the decided histomorphological feature. In this case, the prediction model does not always need to be generated by machine learning, and the user may generate the prediction model (that is, decide a parameter for setting the prediction model).

### <<Kind of Cancer Therapeutic Drug or Side Effect to be Predicted>>

The prediction data 30 is generated for one or more kinds of cancer therapeutic drugs. The information processing apparatus 2000 may be configured to generate the prediction data 30 for a predetermined kind of cancer therapeutic drug in advance or may determine the kind of a cancer therapeutic drug to be predicted and generate the prediction data 30 for the determined kind of cancer therapeutic drug. In the latter case, for example, the information processing apparatus 2000 receives an input operation to specify the kind of a cancer therapeutic drug to be predicted. In this case, the information processing apparatus 2000 generates the prediction data 30 for the specified kind of cancer therapeutic drug. In addition, for example, information indicating the kind of the cancer therapeutic drug to be predicted may be stored in the storage apparatus. In this case, the information processing apparatus 2000 reads information from the storage apparatus, thereby determining the kind of the cancer therapeutic drug to be predicted.

The effect of the cancer therapeutic drug may be predicted without determination of the kind of cancer or may be predicted for a specific kind of cancer. In the latter case, the information processing apparatus 2000 may be configured to generate the prediction data 30 for a predetermined kind of cancer in advance or may determine the kind of cancer to be predicted and may generate the prediction data 30 for the determined kind of cancer. A method of determining the kind of cancer to be predicted is the same as a method of determining the kind of the cancer therapeutic drug to be predicted.

In a case where there are a plurality of kinds of side effects, the prediction data 30 is generated for one or more kinds of side effects. The information processing apparatus 2000 may be configured to generate the prediction data 30 for a predetermined kind of side effect in advance or may determine the kind of a side effect to be predicted and generate the prediction data 30 for the determined kind of side effect. A method of determining the side effect to be predicted is the same as the method of determining the cancer therapeutic drug to be predicted.

### <Output of Prediction Data 30>

The generation unit 2040 outputs the generated prediction data 30 in any form. For example, the generation unit 2040 stores the prediction data 30 in the storage apparatus. In addition, for example, the generation unit 2040 may make a display apparatus display the prediction data 30.

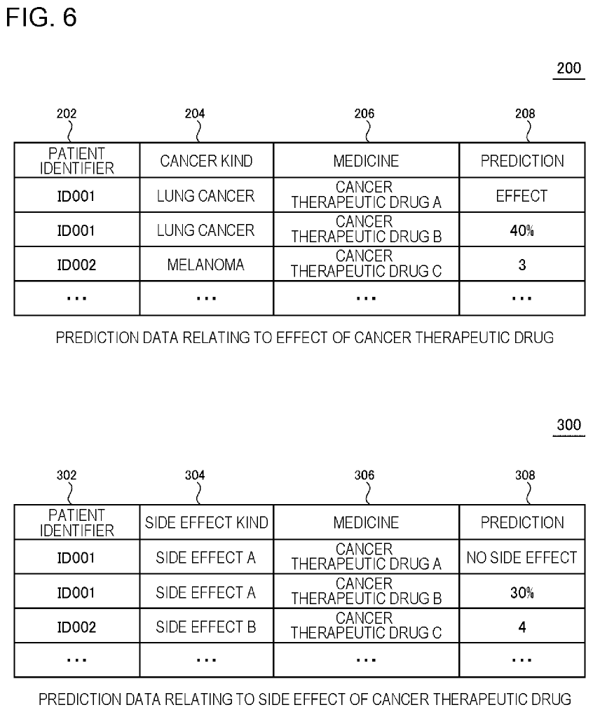

FIG. 6 is a diagram illustrating the prediction data 30 in a table format. A table 200 illustrates the prediction data 30 indicating the prediction relating to the effect of the cancer therapeutic drug. On the other hand, a table 300 illustrates the prediction data 30 indicating the prediction relating to the side effect of the cancer therapeutic drug.

The table 200 has fields of patient identifier 202, cancer kind 204, medicine 206, and prediction 208. The patient identifier 202 is an identifier assigned to a target patient. The cancer kind 204 indicates the kind of cancer to be predicted. The medicine 206 indicates the kind of a cancer therapeutic drug to be predicted. The prediction 208 indicates a prediction relating to an effect of a cancer therapeutic drug. In a first record, the prediction 208 indicates a prediction of the presence or absence of an effect of a cancer therapeutic drug. In a second record, the prediction 208 indicates a prediction of a probability that an effect of a cancer therapeutic drug appears. In a third record, the prediction 208 indicates a prediction of magnitude of an effect of a cancer therapeutic drug with five-grade evaluation.

The table 300 has fields of patient identifier 302, side effect kind 304, medicine 306, and prediction 308. The patient identifier 302 is an identifier assigned to a target patient. The side effect kind 304 indicates the kind of a side effect to be predicted. The medicine 306 indicates the kind of a cancer therapeutic drug to be predicted. The prediction 308 indicates a prediction relating to a side effect of a cancer therapeutic drug. In a first record, the prediction 308 indicates a prediction of the presence or absence of a side effect. In a second record, the prediction 308 indicates a prediction of a probability that a side effect appears. In a third record, the prediction 308 indicates a prediction of magnitude of a side effect with five-grade evaluation.

Although the example embodiment of the invention has been described referring to the drawings, the example embodiment is merely an example of the invention. The invention can employ a combination of the example embodiment or various configurations other than the above.

A part or the whole of the above-described example embodiment can be described as, but is not limited to, the following supplementary notes.

1. An information processing apparatus including:

an extraction unit that extracts a histomorphological feature of a tissue included in pathological image data of a target patient; and

a generation unit that generates, using the extracted histomorphological feature, prediction data indicating one or more of a prediction relating to an effect of a cancer therapeutic drug on the target patient and a prediction relating to a side effect of the cancer therapeutic drug on the target patient.

2. The information processing apparatus described in 1,

in which the cancer therapeutic drug is an immune checkpoint blockade.

3. The information processing apparatus described in 1 or 2,

in which the pathological image data is image data in which a tissue subjected to immunohistochemistry (IHC) is included, and

the extraction unit extracts a histomorphological feature of PD-L1 included in the pathological image data.

4. The information processing apparatus described in 3,

in which the histomorphological feature of the PD-L1 extracted by the extraction unit is one or more of entire circumference of the PD-L1 in a tumor cell with expression of the PD-L1, staining intensity of the PD-L1, and a size of a tumor cell with expression of the PD-L1.

5. The information processing apparatus described in any one of 1 to 4,

in which the pathological image data is image data in which a tissue subjected to immunohistochemistry (IHC) is included, and

the extraction unit extracts a histomorphological feature of an immune cell included in the pathological image data.

6. The information processing apparatus described in 5,

in which the histomorphological feature of the immune cell extracted by the extraction unit is one or more of a positive rate, staining intensity of the immune cell, and a size of the immune cell.

7. The information processing apparatus described in any one of 1 to 6,

in which the pathological image data is image data in which a tissue subjected to hematoxylin and eosin staining is included, and

the extraction unit extracts a histomorphological feature of a cell nucleus of a tumor cell included in the pathological image data.

8. The information processing apparatus described in 7,

in which the histomorphological feature of the cell nucleus extracted by the extraction unit is one or more of an area of the cell nucleus, a perimeter of the cell nucleus, a degree of circularity of the cell nucleus, a degree of complexity of a contour of the cell nucleus, an index value relating to texture of the cell nucleus, a major diameter of the cell nucleus, a minor diameter of the cell nucleus, a ratio of the area of the cell nucleus to an area of a bounding rectangle of the cell nucleus, and density of the cell nucleus.

9. The information processing apparatus described in any one of 1 to 8,

in which the generation unit applies the histomorphological feature extracted by the extraction unit to a prediction model to generate the prediction data, the prediction model having learned to output a prediction relating to an influence of the cancer therapeutic drug on the target patient in response to an input of the histomorphological feature.

10. The information processing apparatus described in any one of 1 to 9,

in which, for a partial region included in the pathological image data, the extraction unit detects a tumor cell from cell nuclei included in the partial region and extracts a histomorphological feature for the cell nucleus of the detected tumor cell or the PD-L1 expressed in the tumor cell.

11. A control method that is executed by a computer, the control method including:

an extraction step of extracting a histomorphological feature of a tissue included in pathological image data of a target patient; and

a generation step of generating, using the extracted histomorphological feature, prediction data indicating one or more of a prediction relating to an effect of a cancer therapeutic drug on the target patient and a prediction relating to a side effect of the cancer therapeutic drug on the target patient.

12. The control method described in 11,

in which the cancer therapeutic drug is an immune checkpoint blockade.

13. The control method described in 11 or 12,

in which the pathological image data is image data in which a tissue subjected to immunohistochemistry (IHC) is included, and

in the extraction step, a histomorphological feature of PD-L1 included in the pathological image data is extracted.

14. The control method described in 13,

in which the histomorphological feature of the PD-L1 extracted in the extraction step is one or more of entire circumference of the PD-L1 in a tumor cell with expression of the PD-L1, staining intensity of the PD-L1, and a size of the tumor cell with expression of the PD-L1.

15. The control method described in any one of 11 to 14,

in which the pathological image data is image data in which a tissue subjected to immunohistochemistry (IHC) is included, and

in the extraction step, a histomorphological feature of an immune cell included in the pathological image data is extracted.

16. The control method described in 15,

in which the histomorphological feature of the immune cell extracted in the extraction step is one or more of a positive rate, staining intensity of the immune cell, and a size of the immune cell.

17. The control method described in any one of 11 to 16,

in which the pathological image data is image data included in a tissue subjected to hematoxylin and eosin staining, and

in the extraction step, a histomorphological feature of a cell nucleus of a tumor cell included in the pathological image data is extracted.

18. The control method described in 17,

in which the histomorphological feature of the cell nucleus extracted in the extraction step is one or more of an area of the cell nucleus, a perimeter of the cell nucleus, a degree of circularity of the cell nucleus, a degree of complexity of a contour of the cell nucleus, an index value relating to texture of the cell nucleus, a major diameter of the cell nucleus, a minor diameter of the cell nucleus, a ratio of the area of the cell nucleus to an area of a bounding rectangle of the cell nucleus, and density of the cell nucleus.

19. The control method described in any one of 11 to 18,

in which, in the generation step, the histomorphological feature extracted in the extraction step is applied to a prediction model to generate the prediction data, the prediction model having learned to output a prediction relating to an influence of the cancer therapeutic drug on the target patient in response to an input of the histomorphological feature.

20. The control method described in any one of 11 to 19,

in which, in the extraction step, for a partial region included in the pathological image data, a tumor cell is detected from cell nuclei included in the partial region and a histomorphological feature is extracted for the cell nucleus of the detected tumor cell or the PD-L1 expressed in the tumor cell.

21. A program causing a computer to execute each step of the control method described in any one of 11 to 20.

This application is based upon and claims the benefit of priority from Japanese patent application No. 2018-085540, filed on Apr. 26, 2018, the disclosure of which is incorporated herein in its entirety by reference.

1. An information processing apparatus comprising:
an extraction unit that extracts a histomorphological feature of a tissue included in pathological image data of a target patient; and
a generation unit that generates, using the extracted histomorphological feature, prediction data indicating one or more of a prediction relating to an effect of a cancer therapeutic drug on the target patient and a prediction relating to a side effect of the cancer therapeutic drug on the target patient.

2. The information processing apparatus according to claim 1,
wherein the cancer therapeutic drug is an immune checkpoint blockade.

3. The information processing apparatus according to claim 1,
wherein the pathological image data is image data in which a tissue subjected to immunohistochemistry (IHC) is included, and
the extraction unit extracts a histomorphological feature of PD-L1 included in the pathological image data.

4. The information processing apparatus according to claim 3,
wherein the histomorphological feature of the PD-L1 extracted by the extraction unit is one or more of entire circumference of the PD-L1 in a tumor cell with expression of the PD-L1, staining intensity of the PD-L1, and a size of a tumor cell with expression of the PD-L1.

5. The information processing apparatus according to claim 1,
wherein the pathological image data is image data in which a tissue subjected to immunohistochemistry (IHC) is included, and
the extraction unit extracts a histomorphological feature of an immune cell included in the pathological image data.

6. The information processing apparatus according to claim 5,
wherein the histomorphological feature of the immune cell extracted by the extraction unit is one or more of a positive rate, staining intensity of the immune cell, and a size of the immune cell.

7. The information processing apparatus according to claim 1,
wherein the pathological image data is image data in which a tissue subjected to hematoxylin and eosin staining is included, and
the extraction unit extracts a histomorphological feature of a cell nucleus of a tumor cell included in the pathological image data.

8. The information processing apparatus according to claim 7,
wherein the histomorphological feature of the cell nucleus extracted by the extraction unit is one or more of an area of the cell nucleus, a perimeter of the cell nucleus, a degree of circularity of the cell nucleus, a degree of complexity of a contour of the cell nucleus, an index value relating to texture of the cell nucleus, a major diameter of the cell nucleus, a minor diameter of the cell nucleus, a ratio of the area of the cell nucleus to an area of a bounding rectangle of the cell nucleus, and density of the cell nucleus.

9. The information processing apparatus according to claim 1,
wherein the generation unit applies the histomorphological feature extracted by the extraction unit to a prediction model to generate the prediction data, the prediction model having learned to output a prediction relating to an influence of the cancer therapeutic drug on the target patient in response to an input of the histomorphological feature.

10. The information processing apparatus according to claim 1,
wherein, for a partial region included in the pathological image data, the extraction unit detects a tumor cell from cell nuclei included in the partial region and extracts a histomorphological feature for the cell nucleus of the detected tumor cell or the PD-L1 expressed in the tumor cell.

11. A control method that is executed by a computer, the control method comprising:
extracting a histomorphological feature of a tissue included in pathological image data of a target patient; and
generating, using the extracted histomorphological feature, prediction data indicating one or more of a prediction relating to an effect of a cancer therapeutic drug on the target patient and a prediction relating to a side effect of the cancer therapeutic drug on the target patient.

12. The control method according to claim 11,
wherein the cancer therapeutic drug is an immune checkpoint blockade.

13. The control method according to claim 11,
wherein the pathological image data is image data in which a tissue subjected to immunohistochemistry (IHC) is included, and
in the extracting, a histomorphological feature of PD-L1 included in the pathological image data is extracted.

14. The control method according to claim 13,
wherein the histomorphological feature of the PD-L1 extracted in the extracting is one or more of entire circumference of the PD-L1 in a tumor cell with expression of the PD-L1, staining intensity of the PD-L1, and a size of a tumor cell with expression of the PD-L1.

15. The control method according to claim 11,
wherein the pathological image data is image data in which a tissue subjected to immunohistochemistry (IHC) is included, and
in the extracting, a histomorphological feature of an immune cell included in the pathological image data is extracted.

16. The control method according to claim 15,
wherein the histomorphological feature of the immune cell extracted in the extracting is one or more of a positive rate, staining intensity of the immune cell, and a size of the immune cell.

17. The control method according to claim 11,
wherein the pathological image data is image data in which a tissue subjected to hematoxylin and eosin staining is included, and
in the extracting, a histomorphological feature of a cell nucleus of a tumor cell included in the pathological image data is extracted.

18. The control method according to claim 17,
wherein the histomorphological feature of the cell nucleus extracted in the extracting is one or more of an area of the cell nucleus, a perimeter of the cell nucleus, a degree of circularity of the cell nucleus, a degree of complexity of a contour of the cell nucleus, an index value relating to texture of the cell nucleus, a major diameter of the cell nucleus, a minor diameter of the cell nucleus, a ratio of the area of the cell nucleus to an area of a bounding rectangle of the cell nucleus, and density of the cell nucleus.

19. The control method according to claim 11,
wherein, in the generating, the histomorphological feature extracted in the extracting is applied to a prediction model to generate the prediction data, the prediction model having learned to output a prediction relating to an influence of the cancer therapeutic drug on the target patient in response to an input of the histomorphological feature.

20. (canceled)

21. A non-transitory computer-readable storage medium storing a program causing a computer to execute the control method according to claim 11.
